(19) United States
(12) Patent Application Publication
(10) Pub. No.: US 2020/0186638 A1
(43) Pub. Date: Jun. 11, 2020

(54) Vehicle Safety and Reminder Device

(71) Applicant: Cathleen Horniak, Whiteville, NC (US)

(72) Inventor: Cathleen Horniak, Whiteville, NC (US)

(21) Appl. No.: 16/702,815

(22) Filed: Dec. 4, 2019

Related U.S. Application Data
(60) Provisional application No. 62/777,309, filed on Dec. 10, 2018.

(51) Int. Cl. H04M 1/725; B60N 2/00; B60N 2/58; H04W 4/48; A61B 5/00; A61B 5/024

(52) U.S. Cl. CPC H04M 1/72538; B60N 2/002; B60N 2/58; H04W 4/48; B60R 16/03; A61B 5/6893; A61B 5/0002; A61B 5/024; A61B 5/746; A61B 5/6892

(57) ABSTRACT

A vehicle safety reminder mat including a rubber mat having an interior volume. The mat houses a heartbeat sensor. Further, the mat houses a pressure sensor. The sensors are connected to a power source. The power source is capable of being connected to a vehicle cigarette lighter. A wireless transceiver is connected to the heartbeat sensor and the pressure sensor. The wireless transceiver and the power source are located in an external box that is connected to the mat. The mat has a reusable adhesive placed on a backside of the mat. The mat will send a signal to a user when the sensors detect a living being and a user has left the vicinity of the mat.

### CROSS REFERENCE TO RELATED APPLICATIONS

This application claims the benefit of U.S. Provisional Application No. 62/777,309 filed on Dec. 10, 2018. The above identified patent application is herein incorporated by reference in its entirety to provide continuity of disclosure.

### BACKGROUND OF THE INVENTION

The present invention relates to a vehicle safety and reminder device. More particularly, the present invention provides a vehicle safety and reminder device that reminds a user if they have left a living being in a vehicle.

Many people use vehicles to drive from place to place. Of these people, many people transport young children or pets. It is common knowledge that the interior of a vehicle can become extremely hot very quickly. In the summer months, the interior of a vehicle can reach temperatures in excess of 120 degrees in minutes. At these rapidly climbing temperatures, children or animals can suffer heat stroke, or even death, in minutes.

Unfortunately, even though it is common knowledge that climbing temperatures in cars can kill small children and animals in minutes, countless people accidentally leave kids and pets locked in cars. Many people are constantly in a hurry and forget to look in the back of the car. However, the same people who forget to look in the back seat of their car also seem to remember their cell phones. The present device is configured to communicate with a cell phone.

Consequently, there is a need in for an improvement in the art of vehicle safety and reminders. The present invention substantially diverges in design elements from the known art while at the same time solves a problem many people face when forgetting to check the backseat of a vehicle when exiting. In this regard the present invention substantially fulfills these needs.

### SUMMARY OF THE INVENTION

The present invention provides a vehicle safety reminder device wherein the same can be utilized for providing convenience for the user when transporting a loved one or pet. The present system comprises a mat having an internal area, wherein the mat is flexible. A heartbeat monitoring sensor is located within the mat. A wireless transceiver is operably coupled to the heartbeat monitoring sensor. A power source is operably coupled to the heartbeat monitoring sensor and the wireless transceiver.

Another object of the present invention is to provide a vehicle safety reminder device, comprising a mat having an internal area, wherein the mat is flexible.

Another object of the present invention is to include an external box to house components for the vehicle safety reminder device.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the vehicle safety and reminder device.

FIG. 2 shows a perspective view of an embodiment of the vehicle safety and reminder device with an external compartment.

FIG. 3 shows a rear view of an embodiment of the vehicle safety and reminder device having an adhesive.

FIG. 4 shows a close-up view of an embodiment of the plug for the safety and reminder device.

FIG. 5 shows a diagram of an embodiment of the wiring for the safety and reminder device.

FIG. 6 shows a flow chart of an embodiment of how the device will function.

### DETAILED DESCRIPTION OF THE INVENTION

Reference is made herein to the attached drawings. Like reference numerals are used throughout the drawings to depict like or similar elements of the vehicle safety and reminder device. For the purposes of presenting a brief and clear description of the present invention, a preferred embodiment will be discussed as used for the reminding a user to check the back seat of a vehicle when they exit for living beings. The figures are intended for representative purposes only and should not be considered to be limiting in any respect.

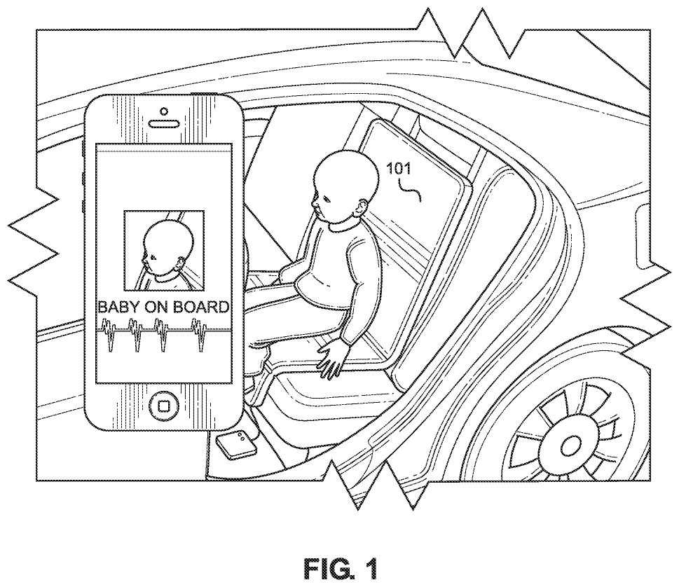

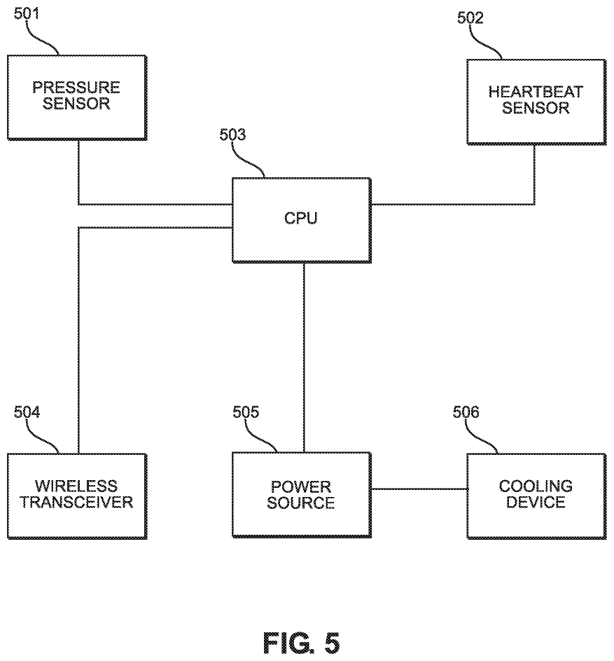

Referring now to FIG. 1, there is shown a perspective view of an embodiment of the vehicle safety and reminder device. The vehicle safety and reminder device is comprised of a flexible mat 101 that houses a plurality of sensors to be detailed in the description of FIG. 5. In one embodiment, the flexible mat 101 is a rubber material, such that it can be easily cleaned while at the same time being a soft surface to provide comfort. In one embodiment, the flexible mat 101 is a foam. Foam is light weight and flexible and easy to fit under or within a car seat or booster seat. The flexible mat 101 is configured to be able to fit in a vehicle seat. The flexible mat 101 is further configured to fit in various children's car seats and booster seats. This is due to the flexibility of the mat. It can be bent to fit in many different areas.

In the illustrated embodiment, the vehicle safety and reminder device is configured to work with mobile devices. In the shown embodiment, the vehicle safety and reminder device will work with an application that is downloaded on a mobile device. The mobile application will have the ability to alert a user if a living being is present. Further, the mobile application is configured to take readings from the various sensors.

In another embodiment, the vehicle safety and reminder device is designed to work with a remote receiver. The remote receive is made to connect to the flexible mat 101 located inside the car. The remote receiver has a speaker and a screen. The remote receiver will be designed to function similar to the mobile device and application. In this embodiment, a mobile device such as a cell phone or tablet will not be required to use the vehicle safety and reminder device.

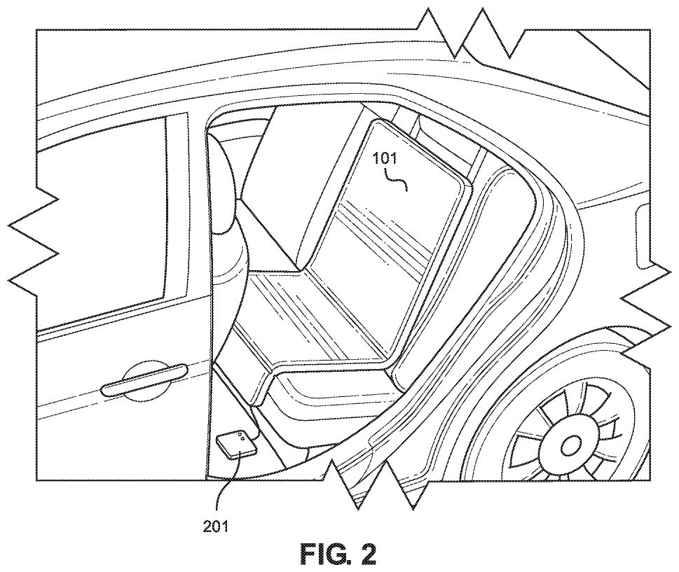

Referring now to FIG. 2, there is shown a perspective view of an embodiment of the vehicle safety and reminder device with an external compartment. In the illustrated embodiment, there is an external compartment 201 that is attached to the flexible mat 101. The external compartment 201 has an internal volume. The external compartment 201 houses a power source within the interior volume. In one embodiment, the power source is a battery. In one embodiment, the power source is a wired power source. In one embodiment, the power source can be a combination of a rechargeable battery and a wired battery charger.

In one embodiment, the external compartment 201 houses a cooling device. In one embodiment, the cooling device is a fan. In one embodiment, the cooling device is an air conditioning unit. The external compartment 201 further houses several other electrical components as described in the description of FIG. 5.

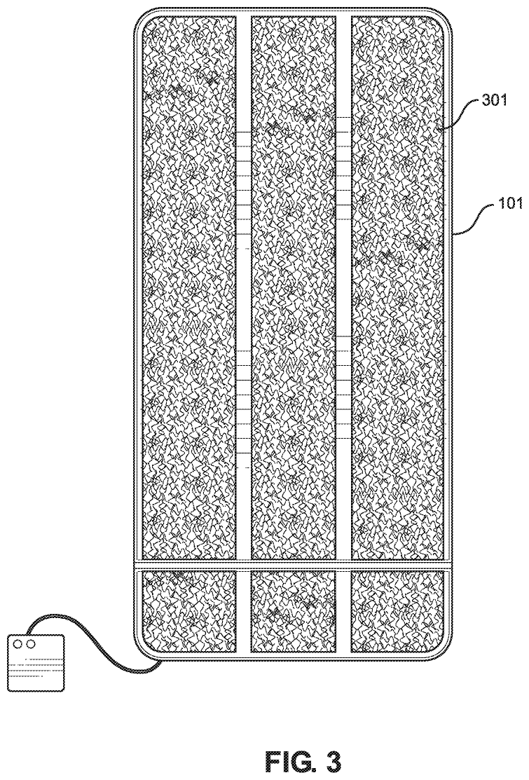

Referring now to FIG. 3, there is shown a rear view of an embodiment of the vehicle safety and reminder device having an adhesive. In one embodiment, the backside of the flexible mat 101 has an adhesive 301 thereon. In one embodiment, the adhesive 301 is a reusable adhesive. In one embodiment, the adhesive is a flexible adhesive. In another embodiment, the adhesive is a gel adhesive.

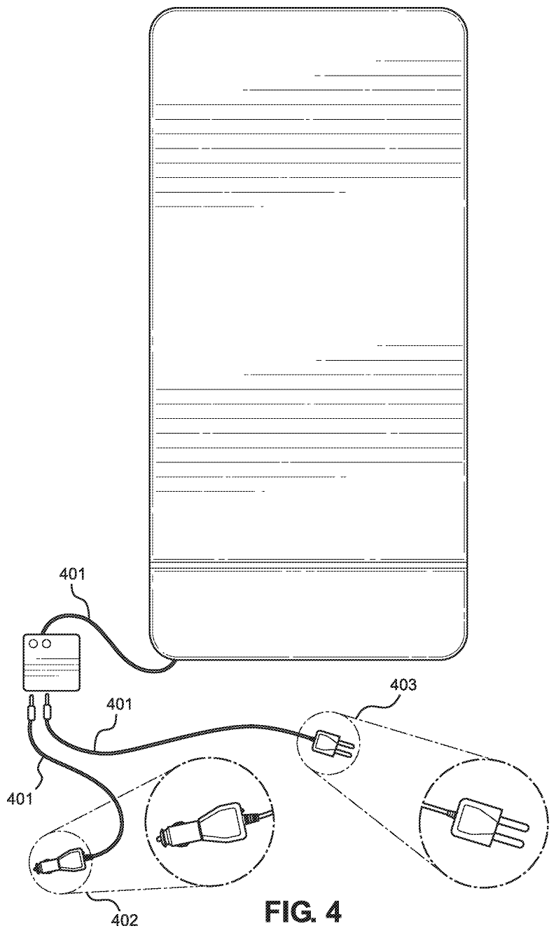

Referring now to FIG. 4, there is shown a close-up view of an embodiment of the plug for the vehicle safety and reminder device. In several embodiments, the vehicle safety and reminder device has the ability to connect directly to a power source. In one embodiment, cord 401 supplies power directly to the vehicle safety and reminder device. In one embodiment, the cord 401 can include a plug that is adapted to use a cigarette lighter 402. In another embodiment, the cord 401 includes a standard wall plug 403. In one embodiment, a combination of the two types of cords 401 is used. In one embodiment, the connection is used as a means to charge the rechargeable battery. In a version of this embodiment, the cord 401 may be removable. This will allow a user to charge the device then remove the cord 401 or cords 401.

Referring now to FIG. 5, there is shown a diagram of an embodiment of the wiring for the safety and reminder device. There is a CPU 503 that operates to control the vehicle device and safety device. The CPU 503 is operably coupled to a power source 505. The power source 505 may be either a battery or a direct power source 505 or a combination of the two. The power source 505 is operably coupled to a cooling device 506. The CPU 503 is further operably coupled to a wireless transceiver 504. In one embodiment, the wireless transceiver 504 is configured to support Bluetooth. In one embodiment, the wireless transceiver 504 is configured to support WIFI.

The CPU 503 is connected to a plurality of sensors. In one embodiment, there is at least one pressure sensor 501. The at least one pressure sensor 501 is configured to detect when weight is placed on the mat. In one embodiment, there is a plurality of thin flexible pressure sensors 501 placed within the mat. The CPU 503 is further operably coupled to a heartbeat sensor 502. The heartbeat sensor 502 is configured to be placed within the mat. Further, the heartbeat sensor 502 is configured to be sensitive enough to be able to detect a heartbeat through the mat and without any direct contact with an artery. In this configuration, the sensor will be able to detect a heartbeat when a living being is placed on the mat.

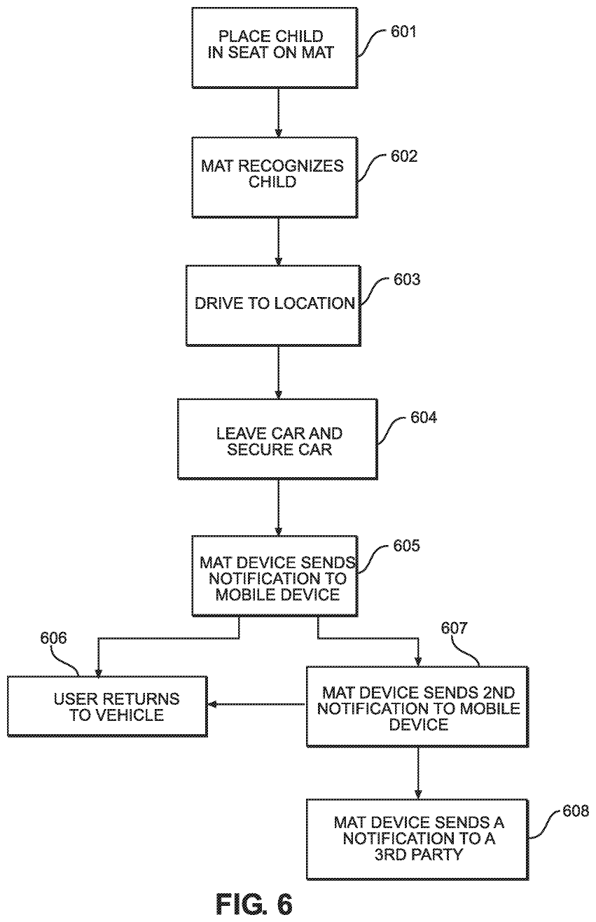

Referring now to FIG. 6, there is shown a flow chart of an embodiment of how the device will function. In one embodiment, the method starts when a child or other living being is placed on the flexible mat of the vehicle safety and reminder device 601. The flexible mat will then use the sensors to recognize the living being 602. The user of the vehicle safety and reminder device will then move the vehicle to a desired location 603. In this embodiment, the user will then leave the vehicle and secure the vehicle 604. The user will not have remembered to remove the living being from the vehicle.

After a set amount of time the vehicle safety and reminder device will send a first reminder to the user 605. In one embodiment, the reminder will be sent to a mobile device. In an embodiment, the notification is sent 10 seconds after the car is secured. In one embodiment, the user immediately returns to the vehicle and the method ends. If the user does not return to the vehicle after a set amount of time, a second reminder is sent 607. In one embodiment, the reminder will be sent to a mobile device. In an embodiment, the notification is sent 10 seconds after the car is secured. In another embodiment, the notification is sent after 15 seconds. In one embodiment, the user returns to the vehicle and the method ends. In another embodiment, more notifications can be sent after a set amount of time.

In one embodiment a further notification is sent to a third party 608. In this embodiment a after the second notification is send to the mobile device 607. After this notification is send the user is given a set amount of time to return to the vehicle ending the cycle 606. If the user does not return to the vehicle in this amount of time then a third party is notified 608. In one embodiment the third party is a special agency. In another embodiment the third party is another registered user.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A vehicle safety and reminder device, comprising:
a mat having an internal area, wherein the mat is flexible;
a heartbeat monitoring sensor located within the mat;
a wireless transceiver operably coupled to the heartbeat monitoring sensor;
a power source operably coupled to the heartbeat monitoring sensor and the wireless transceiver.

2) The vehicle safety and reminder device of claim 1, further comprising a reusable adhesive located on a backside of the mat.

3) The vehicle safety and reminder device of claim 1, further comprising an external box, wherein the external box has an internal volume.

4) The vehicle safety and reminder device of claim 3, wherein the power source and the wireless transceiver is located within the external internal volume.

5) The vehicle safety and reminder device of claim 1, wherein the power source includes at least a rechargeable battery.

6) The vehicle safety and reminder device of claim 1, wherein the mat is made from rubber.

7) The vehicle safety and reminder device of claim 1, wherein the power source is a vehicle connector.

8) A vehicle safety and reminder device, comprising:
a mat having an internal area, wherein the mat is flexible;
a heartbeat monitoring sensor located within the mat;
a pressure sensor located within the mat;
a wireless transceiver is operably coupled to the heartbeat monitoring sensor;
a power source is operably coupled to the heartbeat monitoring sensor and the wireless transceiver.

9) The vehicle safety and reminder device of claim 1, further comprising a reusable adhesive located on a backside of the mat.

10) The vehicle safety and reminder device of claim 1, further comprising an external box, wherein the external box has an internal volume.

11) The vehicle safety and reminder device of claim 3, wherein the power source and the wireless transceiver is located within the external box.

12) The vehicle safety and reminder device of claim 1, wherein the power source includes at least a rechargeable battery.

13) The vehicle safety and reminder device of claim 1, wherein the mat is made from rubber.

14) The vehicle safety and reminder device of claim 1, wherein the power source is a vehicle connector.

15) A method for a vehicle safety reminder, the method comprising:
Recognizing a child on a flexible weight sensing mat within a vehicle;
Determining that the vehicle has been locked;
Sending a notification to a device that the child is still on the weight sensing mat.

16) The method for a vehicle safety reminder of claim 15 further comprising, Sending a second notification to a device that the child is still on the weight sensing mat.

17) The method for a vehicle safety reminder of claim 15 wherein the notification is sent after 10 seconds.

18) The method for a vehicle safety reminder of claim 16 wherein the notification is sent after 10 seconds.

19) The method for a vehicle safety reminder of claim 16 wherein the notifications are sent after a predetermined time interval.
